(19) United States
(12) Patent Application Publication
(10) Pub. No.: US 2008/0125863 A1
(43) Pub. Date: May 29, 2008

(54) Implant designs and methods of improving cartilage repair

(76) Inventor: William F. McKay, Memphis, TN (US)

Correspondence Address: FOX ROTHSCHILD, LLP, 997 LENOX DRIVE, LAWRENCEVILLE, NJ, 08648, US

(21) Appl. No.: 11/605,116

(22) Filed: Nov. 28, 2006

(51) Int. Cl. A61F 2/02; A61F 2/28; A61B 17/08

(52) U.S. Cl. 623/11.11; 623/23.63; 623/23.72; 606/151

(57) ABSTRACT

The invention herein generally refers to an in situ cartilage repair implant. The implant promotes cartilage repair by providing a sealed barrier that prevents the flow of synovial fluid and inflammatory cytokines into a surgically prepared hole that accommodates the implant. Optionally, additives are associated with the implant to promote cartilage repair.

### FIELD OF INVENTION

An in situ cartilage repair implant is disclosed. More specifically, a device is disclosed that comprises a scaffold crowned with a protective cover. The implant promotes cartilage repair by providing a sealed barrier that prevents the flow of synovial fluid and inflammatory cytokines located in the synovial cavity into a surgically prepared defect that accommodates the implant. Optionally, additives are associated with the implant to induce cartilage repair.

### BACKGROUND OF THE INVENTION

Hyaline cartilage is connective tissue found in parts of the body where support, flexibility, and resistance to compression are desired, (e.g. the tip of the nose, and also the ends of bone-forming joints). Hyaline cartilage consists of cells called chondrocytes, which are embedded in a highly specialized extra-cellular matrix. Hyaline cartilage is lubricated with a viscous fluid, called synovial fluid, found in and about articular joints. Normal synovial fluid contains hyaluronic acid, polymeric disaccharides, and lubricin. Together, the synovial fluid and hyaline cartilage act as a shock absorber, and reduce friction to permit bones to move smoothly over one another.

Degenerative diseases wear away hyaline cartilage covering the end of bones, causing inflammation-related pain, swelling, bone spur formation and decreased motion. Millions of people in the United States and throughout the world are affected by bone degenerative diseases, which may include osteoarthritis, osteoporosis, Paget's disease, and osteohalisteresis. These diseases often necessitate joint replacement surgeries, cartilage replacement procedures and the like. For instance, it is estimated that in the United States 650,000 reparative knee procedures affecting hyaline cartilage are carried out each year.

Any trauma or frequent strain on joints causing damage to hyaline cartilage will heal slowly or with serious defects to the repaired tissue. This is due in part to hyaline cartilage being avascular, lacking the nerves, blood vessels and lymphatic systems that facilitate healing. The cartilage repair process is further slowed by synovial fluid and inflammatory cytokines that travel from the synovial cavity into the defect where cartilage and sub-chondral bone tissue are undergoing repair.

It is believed that the glycoprotein lubricin found in synovial fluid reduces the integrative repair capacity of cartilage (see Schaefer, D. B. et al., “Lubricin Reduces Cartilage-Cartilage Integration,” Biorhelogy, vol. 41. IOS Press, pp. 503-508, 2004). In addition, inflammatory cytokines stimulate chondrocytes to produce certain proteins that inhibit the synthesis of type II collagen needed for hyaline cartilage repair.

Typically, when hyaline cartilage heals, it lacks the structural and physical properties of healthy cartilage (fibrocartilage) and will degenerate over time. If the injury is not properly treated, it can progress into a degenerative disease. Proper repair of cartilage defects usually requires orthopedic surgery. Patients with damaged hyaline cartilage can opt to have the defective tissue replaced with allografts, prosthetic implants, or new cartilage stimulated by chondrocytes or growth factors isolated in a natural or artificial support.

For example, mosaicplasty procedures use an artistic arrangement of osteochondral implants to heal defective cartilage by boring holes in the base of damaged cartilage and the underlying sub-chondral bone. The holes are filled with autologus cylindrical plugs made of bone and cartilage tissues in a mosaic fashion. However, mosaicplasty can be compromised if the donor cartilage is diseased, if there is damage to the collagen-forming chondrocytes, or if there is a wearing of the graft over time.

Another procedure for treating damaged cartilage involves transplanting large segments of bone and articular cartilage to a damaged joint. A drawback to this procedure is that there must be a fresh donor and the tissue must be stored at low temperatures and used within a month to ensure a greater than 50% cell viability.

Arthroscopic debridement and lavage removes degenerative cartilage debris from the damaged area by irrigating the joint with a salt and lactate solution. These methods provide temporary relief of pain but do little for the formation of new cartilage tissue.

Microfracture procedures involve puncturing small holes into the subchondral bone to induce bleeding. A blood clot is formed when blood and bone marrow seep onto the damaged cartilage, which releases cartilage-building stem cells. Like arthroscopic debridement and lavage, microfracture procedures produce cartilage tissue that is fibrous in nature and degenerates over time.

The prior art discloses devices or gels to treat and repair damaged cartilage. U.S. Pat. Nos. 6,852,125; 6,632,246 and 6,626,945 disclose artificial cartilage repair plugs used individually or in combination with other plugs. The plugs are inserted into voids left by the removal of diseased cartilage by the surgeon. They are made from a biocompatible artificial material, have varying layered and bridged configurations, and can have a plurality of anchoring elements. Certain embodiments have the plugs as anchors for a flowable polymer used to fill a void in the cartilage defect and the sub-chondral bone.

U.S. Pat. No. 7,067,123 discloses a gel for cartilage repair. The gel is a mixture of milled allograft cartilage, a bio-absorbable material, and optional additives. The gel is placed in a lesion or defect that has been removed by boring and then it is fixed in place with a periosteal cap.

U.S. Pat. No. 6,743,232 discloses a device that is anchored into the sub-chondral bone for cartilage repair. The device has a platform for holding a tissue sample, for example an allograft of cartilage. A post extends from the platform and anchors the platform into bone tissue by ribs with sharp edges that are attached to the post.

U.S. Pat. No. 6,582,471 discloses a device for cartilage repair having a porous bio-degradable matrix associated with a composition for in vivo cartilage repair, wherein the device is placed in a cartilage defect. The composition is a mixture derived from bone, cartilage, tendon, meniscus or ligament or a synthetic mimic of such a mixture encapsulated in nano-spheres.

U.S. Pat. No. 7,041,641 discloses a cartilage repair plug that involves admixing growth factors of constant concentration in various matrices to enhance cartilage repair.

U.S. Pat. No. 6,575,986 discloses a scaffold fixation device for use in articular cartilage repair. The device has a platform with a post that extends from the platform and is inserted into a hole formed in the bone. The post has various configurations of ribs that extend from the side surfaces of the post. The device fastens an articular cartilage scaffold to underlying bone tissue.

U.S. Pat. No. 6,514,514 discloses a device and method for regeneration and repair of cartilage lesions. The device is a cartilage repair matrix in the shape of a sheet. The device can be cut or shaped to fit cartilage tears of various shapes and sizes and to cover the entire surface of the damaged tissue. The repair matrix is associated with cartilage inducing compositions made of various chondrogensis-enhancing proteins.

U.S. Pat. No. 5,632,745 discloses a method for surgically implanting a bio-absorbable cartilage repair system into a cartilage defect.

U.S. Pat. No. 6,371,958 provides for a scaffold fixation device, which fastens an articular cartilage scaffold to underlying bone.

U.S. Pat. No. 6,468,314 discloses a bio-absorbable cartilage repair system that allows for vascular invasion and cellular migration between the system and the healthy area of articular cartilage and bone.

Previous attempts to heal hyaline cartilage defects alone have resulted in sub-optimal healing of both the cartilage and bone layers. Often, resorption pits in the sub-chondral bone have been seen and poor resurfacing of the hyaline cartilage is observed. Also, when new hyaline cartilage is seen, it often does not attach to adjacent host hyaline cartilage. Accordingly, there is a need for an implant that effectively promotes cartilage repair by stopping or slowing the influx of synovial fluids and inflammatory cytokines from the synovial cavity into the defect.

### SUMMARY OF THE INVENTION

The present invention overcomes the drawbacks of prior art by providing a novel cartilage implant that comprises a cover, a cartilage repair scaffold, a means for axially fixing the cover to a scaffold end surface, and optionally a gasket. The implant may prevent the influx of synovial fluid and inflammatory cytokines from the synovial cavity into a surgically prepared defect meant for cartilage repair.

In certain embodiments, the cover extends beyond the boarders of the cartilage defect to overhang adjacent normal cartilage surfaces.

In certain specific embodiments, the cover extends beyond the diameter of the scaffold.

In various embodiments, the means for axially fixing the cover to a scaffold end surface includes an anchor, pins, an adhesive, a suture or combinations thereof.

In some embodiments, the anchor axially extends from at least one surface of the cover.

In certain specific embodiments, the anchor is barbed about its exterior.

In other specific embodiments, the anchor is centrally attached to at least one surface of he cover by a glue, staples, a pin, or combinations thereof.

In various other embodiments, the anchor axially fixes the cover to a scaffold end surface.

In other specific embodiments, the anchor is centrally attached to the cover and axially forced through a scaffold end surface, such that the anchor engages the inside surfaces of the cartilage repair scaffold creating a securing interaction between the two.

In various embodiments, the cover, the scaffold, the anchor, the pins or the gasket are made from materials selected from collagen, hyaluronic acid, chitosan, natural polymers, aliphatic polyesters, polyorthoesters, polyanhydrides, polycarbonates, polyurethanes, polyamides, polyalkylene oxides, absorbable polymers, glasses or ceramics, autograft or allograft cartilage tissue, or combinations thereof.

In certain preferred embodiments, the implant, once inserted into the defect, forms a sealed barrier between the outer biochemical environment of the synovial cavity and inner biochemical environment of a surgically prepared defect that extends from the surfaces of hyaline cartilage into sub-chondral bone.

In certain specific embodiments, the cover may be aligned with or slightly below the upper surface of hyaline cartilage of an articular joint after the implant is completely inserted into the surgically prepared defect that extends from the surfaces of hyaline cartilage into sub-chondral bone.

In various embodiments, cover prevents the influx of synovial fluid and inflammatory cytokines into a surgically prepared defect that extends from the surfaces of hyaline cartilage into sub-chondral bone.

In some embodiments, the cover is a sheet.

In some other embodiments, the scaffold is porous.

In other embodiments, the scaffold is non-porous.

In certain embodiments, the scaffold may be bio-resorbable

In other, specific embodiments, additives are associated with the scaffold, the cover, or the gasket.

In certain of these embodiments, the additives are growth factors, antibiotics, analgesics, radiocontrast agents, porogens, anti-inflammatory agents or combinations thereof.

In specific embodiments, at least one growth factor is BMP-1, BMP-2, rhBMP-2, BMP-3, BMP-4, rhBMP-4, BMP-5, BMP-6, rhBMP-6, BMP-7 [OP-1], rhBMP-7, BMP-8, BMP-9, BMP-10, BMP-11, BMP-12, BMP-13, BMP-14, BMP-15, BMP-16, BMP-17, BMP-18, Growth and Differentiation Factors, GDF-5, Cartilage Derived Morphogenic Proteins, LIM mineralization protein, platelet derived growth factor (PDGF), transforming growth factor α, (TGF-α), insulin-related growth factor-I (IGF-I), insulin-related growth factor-II (IGF-II), fibroblast growth factor (FGF), beta-2-microglobulin (BDGF II), or rhGDF-5.

In various embodiments, the scaffold, the cover, or the gasket may have shapes that include cylindrical, oval, square, rod-like, star-shaped or combinations thereof.

In some embodiments, after the scaffold has been inserted into the defect, the cover is axially attached to the scaffold end surface exposed to the synovial cavity.

In other embodiments, before the scaffold is inserted into the defect, the cover is axially attached to the scaffold end surface exposed to the synovial cavity.

In certain preferred embodiments, the cover has a larger surface area than the scaffold end surface to which it is attached.

In some embodiments, at least one cover surface has a shoulder or retaining ridges.

In other embodiments, the cover and at least one scaffold end surface are fixed together with an adherent sealant.

In certain specific embodiments, the cover is an adherent sealant.

In another embodiment, the adherent sealant is disposed on the scaffold end surface that is exposed to the synovial cavity after the scaffold has been inserted into the defect.

In other embodiments, the adherent sealant is cyanoacrylates, methylacrylates, octylacrylates, PEG, glycosaminoglysan, chitosan, collagen, hyaluronic acid, polyurethane solvents, or visible and UV activated adhesives.

In some embodiments, the adherent sealant sticks to surrounding hyaline cartilage tissue long enough to allow proper healing to occur in the areas of the scaffold in contact with both bone and cartilage tissue.

In other aspects, a method for repairing damaged cartilage is provided comprising: i) surgically removing damaged cartilage; ii) drilling a hole in the area of the removed cartilage into sub-chondral bone tissue; iii.) inserting the cartilage repair scaffold into the hole; and iv.) axially securing the cover onto the end surface of the cartilage repair surface that is exposed to the hyaline cartilage region such that a sealed barrier is formed between the outer biochemical environment of hyaline cartilage and inner biochemical environment of the defect. Alternatively, the cover can also be applied to the scaffold at the time of manufacture by “welding” at least one cover surface and a scaffold end surface together via various manufacturing methods known in the art.

### BRIEF DESCRIPTION OF THE DRAWINGS

FIG. 1 is a perspective view of a first embodiment implant.

FIG. 2 is a perspective view of a second embodiment implant.

FIG. 3 is a perspective view of a third embodiment implant.

FIG. 4 is a perspective view of a fourth embodiment implant.

FIG. 5 is a cross-sectional view showing a fifth embodiment implant disposed within a defect.

FIG. 6 is a cross-sectional view showing a sixth embodiment implant disposed within a defect.

FIG. 7 is a cross-sectional view showing a seventh embodiment implant disposed within a defect.

FIG. 8 is a cross-sectional view showing an eighth embodiment implant disposed within a defect.

FIG. 9 is an exploded view of a ninth embodiment implant.

### DETAILED DESCRIPTION OF THE INVENTION

### Definitions

“Scaffold” or “cartilage repair scaffold” generally refers to an implant body or member that may be inserted into a surgically prepared defect. The scaffold acts as a support for the surrounding cartilage and bone tissue of the defect. When attached to the cover, the scaffold may axially extend from the same. The size and shape of the scaffold may depend upon the dimensions of the cartilage defect that needs repairing and the dimensions of the defect that extends into sub-chondral bone.

“Cover” generally refers to a shaped piece of material that may be placed or fixed on a scaffold end surface. The cover may be an oval or otherwise shaped sheet, which when placed or fixed on a scaffold end surface, overhangs the scaffold body and makes contact with hyaline cartilage in or about an articular joint. “Anchor” generally refers to any suitable material that secures the cover to an end surface of the cartilage repair scaffold. The anchor may axially extend from a cover surface and may be axially forced through a scaffold end surface such that the cover is secured to the scaffold end surface. The anchor may engage the inner material of the scaffold to create a secured interaction between the two, and can take many shapes, such as rod-like, pin-like, and so forth. In other embodiments, the anchor may be an adhesive adapted to secure the cover to the scaffold.

“Hyaline cartilage” and “cartilage” generally refer to healthy cartilage in the area near an articular joint where damaged cartilage was surgically removed.

“Replacement procedure” refers to the surgical procedure of removing damaged cartilage from an articular joint, drilling a hole into sub-chondral bone tissue below the area of removed cartilage, and filling the hole with the implant.

“Synovial cavity” generally refers to the space that separates opposing bones that are covered with hyaline cartilage. The synovial cavity is encapsulated by the fibrous joint capsule and is filled with synovial fluid secreted by the synovial membrane.

“Defect” generally refers to a surgically prepared hole that extends from the surfaces of hyaline cartilage into sub-chondral bone tissue.

“Crowned” generally refers to the process of axially fixing the cover to a scaffold end surface, where the cover can be axially fixed to a scaffold end surface by pins, barbed anchors, and the like or by disposing an adhesive on one side of the cover or a scaffold end surface and sealing the two together by the natural curing process of the adhesive.

“Growth factors,” “Bone Morphogenic Proteins,” or “BMPs” may include a class of proteins that induces the growth of new endochondral bone or new hyaline cartilage tissue by morphogenic events. An example of a non-limiting selection of BMPs is BMP-1, BMP-2, rhBMP-2, BMP-3, BMP-4, rhBMP-4, BMP-5, BMP-6, rhBMP-6, BMP-7[OP-1], rhBMP-7, BMP-8, BMP-9, BMP-10, BMP-11, BMP-12, BMP-13, BMP-14, BMP-15, BMP-16, BMP-17, BMP-18, Growth and Differentiation Factors, GDF-5, Cartilage Derived Morphogenic Proteins, LIM mineralization protein, platelet derived growth factor (PDGF), transforming growth factor α,(TGF-β), insulin-related growth factor-I (IGF-I), insulin-related growth factor-II (IGF-II), fibroblast growth factor (FGF), beta-2-microglobulin (BDGF II), and rhGDF-5.

“Pores” and “porous exterior” generally implies an implant having a chemical arrangement that is permeable, allowing for ingrowth of bone and cartilage tissue into the implant and for the passing of additives into the defect.

Various embodiments of the invention are further detailed herein. Although the present invention is primarily intended to treat and repair cartilage lesions, there are no intentions for the use of these words to limit the scope of the invention.

Any and all use of specific language and references are for detailing different embodiments of the same. In addition, and despite explicit reference to only the following embodiments, any and all alterations and further modifications of the invention, as would occur to one having ordinary skill in the art, are intended to be within the scope of the invention.

### A GENERAL EMBODIMENT OF THE INVENTION

The Scaffold, Cover and Anchor

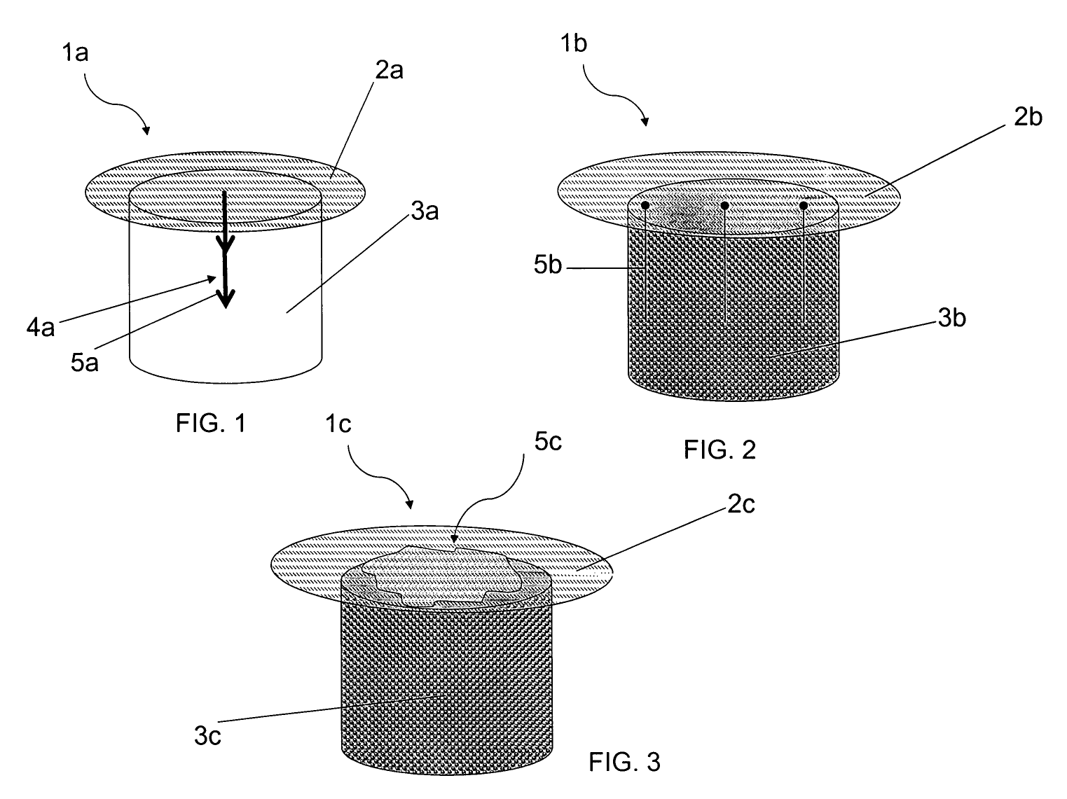

FIGS. 1-4 show a general three-piece embodiment of the invention. Referring to FIG. 1, an implant la typically comprises a protective cover 2a, a cartilage repair scaffold 3a, and a means 4a for axially fixing the cover 2a to the scaffold 3a. In FIG. 1, the cover 2a is axially fixed to the scaffold 3a by a barbed anchor 4a. The anchor 4a is shown as a rod-like structure with vertically protruding barbs 5a about the its exterior.

The anchor 4a may be countersunk into the scaffold 3a such that the barbs 5a, when subjected to an axial force, engage the inner material of the scaffold 3a creating a securing interaction between the two. This embodiment 1a of the invention has the non-barbed end of the anchor 4a axially attached to the center of the cover 2a. The barbs 5a may be added to a basic cylindrical rod by machining, gluing, or by fusing the barbs 5a to the surfaces of the rod. Any suitable method may be employed for attaching the anchor 4a to the cover 2a; for example, the anchor 4a may be glued, stapled, cemented, pinned, and the like, to the underside of the cover 2a that will be in contact with a scaffold 3a end surface.

FIG. 1 shows the implant 1a having a cylindrically shaped scaffold 3a that is non-porous. The scaffold 3a may have a uniform size with circular end surfaces that are connected by a single continuous cylindrical surface between the end surfaces. The cover 2a is depicted as a flat ovoid sheet, wherein the cover 2a may be cut from a various synthetic materials, an allograft of cartilage, or the like. Although FIG. 1 depicts the invention as having a non-porous scaffold 3a, it is within the scope of the invention to have a porous scaffold 3a as well.

FIGS. 2 and 3 depict two non-limiting examples of how one may fix the cover to the scaffold. In FIG. 2, an implant 1b includes a cover 2b that is axially attached to a scaffold 3b end surface by rod like pins 5b, where the pins 5b are axially forced thorough the cover 2b, thereby piercing into scaffold 3b end surface and into its interior.

The size and type of pins 5b used to attach the cover 2b to the scaffold 3b may be at the discretion of the surgeon or given to the surgeon in a prefabricated form. However, the pins 5b should be made of a material capable of piercing and securing into the scaffold material 3b. In FIG. 2, the implant 1b has a porous scaffold 3b. However, it is within the scope of the invention to have a non-porous scaffold 3b as well. It should also be noted that the cover, as shown in all figures, is drawn as transparent for illustrative purposes only.

In FIG. 3, a cover 2c is attached to a porous scaffold 3c end surface with an adhesive 5c. For this embodiment of the invention, a surgeon may spread a thin layer of the adhesive 5c to the underside of the cover 2c that will be in contact with the scaffold 3c end surface. Suitable adhesives 5c may include, but are not limited to, cyanoacrylates, methylacrylates, octylacrylates, polyurethane solvents, or visible and UV activated adhesives. After applying a thin and even layer of adhesive 5c on at least one cover 2c surface, the surgeon may press the cover 2c surface having the adhesive 5c and a scaffold 3c end surface together. After the implant 1c is inserted into the defect, the adhesive's 5c natural curing process ensures that both surfaces remain in contact.

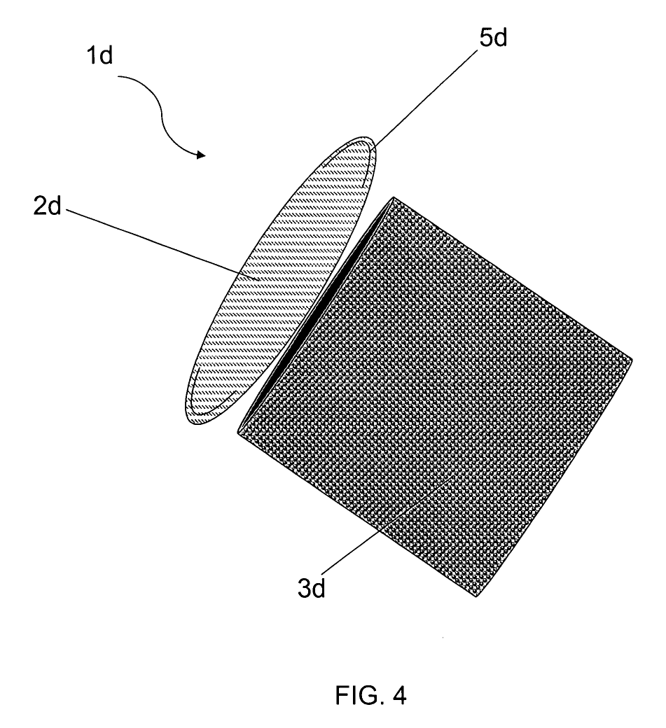

In FIG. 4, a cover 2d further comprises retaining ridges 5d. The retaining ridges 5d may be glued, stapled, cemented, pinned, and the like, to the underside of the cover 2d that will be in contact with the end surface of the scaffold 3d, or may be monolithically formed with the cover 2d. To attach the cover 2d to the scaffold 3d, the retaining ridges 5d are pressed into or around the scaffold 3d. The retaining ridges 5d are preferably placed on the underside of the cover 2d to ensure that the topside of the cover 2d is as smooth as possible to reduce friction. Alternatively, the retaining ridges 5d can be positioned on the outside of the scaffold 3d to form a securing interaction.

Friction between the retaining ridges 5d and the scaffold 3d keeps the cover 2d attached to the scaffold 3d. To increase the friction between the retaining ridges 5d and the scaffold 3d, the size, shape or quantity of the ridges 5d may be varied. For example, the retaining ridges 5d may include barbs similar to barbs 5a shown in FIG. 1. Alternatively, the retaining ridges 5d may be made longer or may extend for substantially the entire circumference of the cover 2d. Although FIG. 4 depicts the implant 1d as having a porous scaffold 3d, it is within the scope of the invention to utilize the retaining ridges 5d to attach the cover 2d to a non-porous scaffold as well.

The Scaffold in the Defect

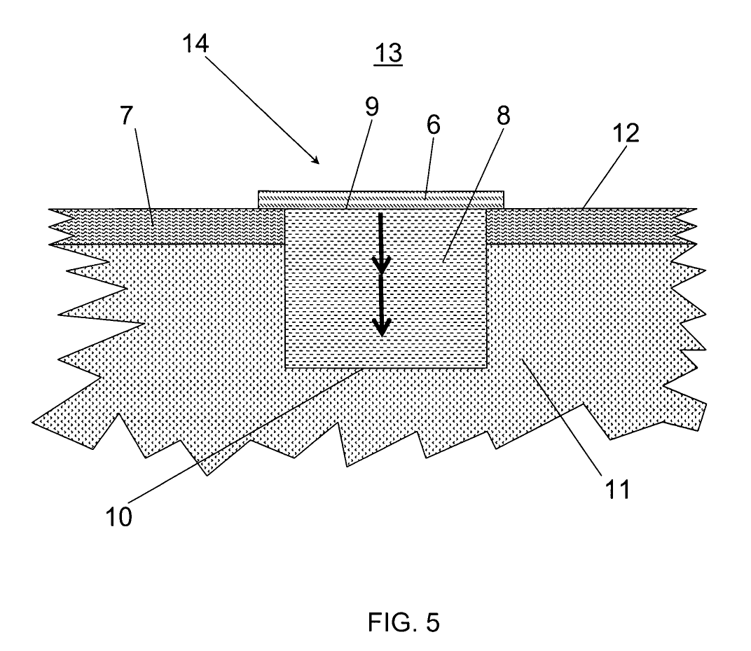

FIG. 5 is a cross-sectional view showing an implant 14 when inserted into a surgically prepared defect 10. The defect 10 extends from the surface 12 of resected hyaline cartilage 7 into sub-chondral bone tissue 11. The dimensions of the defect 10, for example the depth, the diameter, and the various shapes that the defect 10 may take, are based on the size and shape of damaged hyaline cartilage 7 removed by the surgeon.

Preferably, the defect 10 has a diameter that is equal to, or slightly larger than, the outermost diameter of the cartilage repair scaffold 8, such that when the implant 14 is inserted completely into the defect 10 the scaffold 8 is flush against sub-chondral bone tissue 11. The cover 6 covers the upper end surface 9 of the scaffold 8, and additionally ideally overlaps the surrounding surface 12 of hyaline cartilage 7 around the defect 10 exposed to the synovial cavity 13. This implant 14 arrangement creates a sealed barrier between the defect 10 and the biochemical environment of the synovial cavity 13 and the surgically prepared defect 10. The scaffold 8 may be press fitted into the defect 10 or, alternatively, it can be glued into the defect 10 with a biocompatible biodegradable adhesive.

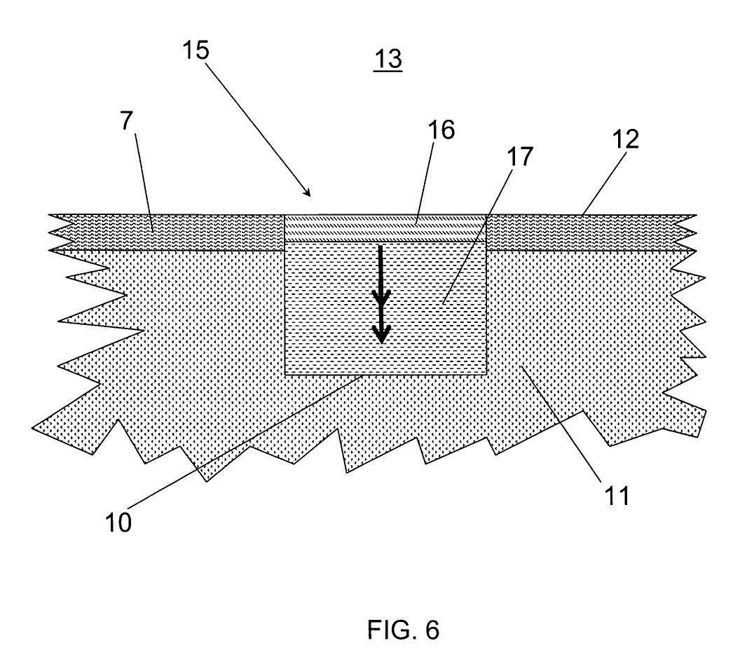

As shown in FIG. 6, in an alternative embodiment, a cover 16 of an implant 15 does not overlap the surrounding hyaline cartilage 7, but is flush with, or very slightly below, the top surface 12 of the cartilage 7. The cover 6 forms a seal with the cartilage 7, and both seals and covers the scaffold 17 of the implant 15.

The cover 6, 16 prevents the passing of synovial fluids and/or inflammatory cytokines from the synovial cavity 13 into the defect 10. In particular, the cover may prevent lubricin within the synovial cavity from passing into the scaffold. It is anticipated that trauma to the hyaline cartilage 7 and the sub-chondral bone 11 caused by the replacement procedure will trigger a heavy macrophage inflammation response. The inflammation response, along with the proteins of the synovial fluid, may slow the reparative process between the implant 14, 15 and the natural cartilage 7 if it were allowed to interact with the same. In any event, the sealed barrier created by the implant 14, 15 enables new tissue growth to occur in and near the inner surfaces of the defect 10 without interference from the like.

The cartilage repair scaffold 8, 17 and the cover 6, 16, can have a range of shapes and sizes, depending on the dimensions of the surgically prepared defect 10 in relation to the dimensions and amount of hyaline cartilage 7 that is removed during the replacement procedure. For example, the scaffold 8, 17 or the cover 6, 16 can have shapes ranging from oval, to cylindrical, to square, to rod-like, or to star shaped just to name a few. The scaffold 8, 17 or the cover 6, 16 may additionally have irregular shapes.

To provide adequate contact, and ideally overlapping contact, between the cover 6, 16 and the surrounding cartilage tissue 7 of the articular joint, the shape and size of both the scaffold 8, 17 or the cover 6, 16 may be determined by the surgeon performing the replacement procedure. Alternatively, the implant 14, 15 can be provided to the surgeon for implantation in a pre-fabricated, off-the-shelf, form, where the shape and size of the implant 14, 15 has been predetermined by someone other than the surgeon performing the replacement procedure.

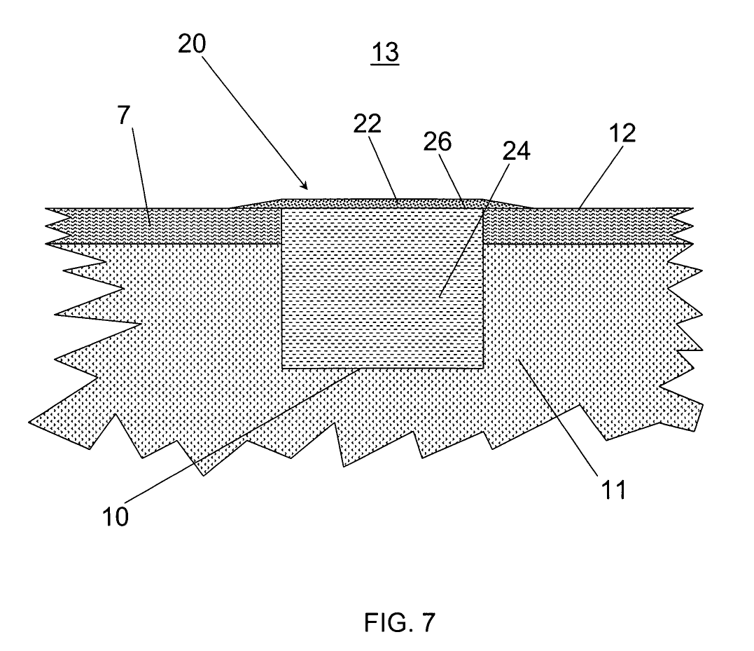

FIG. 7 shows an implant 20 wherein the cover is an adhesive 22. After the scaffold 24 has been inserted into the defect 10, the scaffold 24 is crowned by an adhesive 22 that is disposed over the end surface 26 of the scaffold 24 that would otherwise be exposed to the synovial cavity 13. Enough adhesive 22 is ideally applied to cover the surface area of the scaffold 24 end surface 26 and the surrounding cartilage 12, such that a sealed barrier is formed between the biochemical environment of the synovial cavity 13 and the surgically prepared defect 10 that extends into sub-chondral bone 11. Suitable adhesives 15 are similar to the list of adhesives that can fix the cover to the scaffold as shown in FIG. 3.

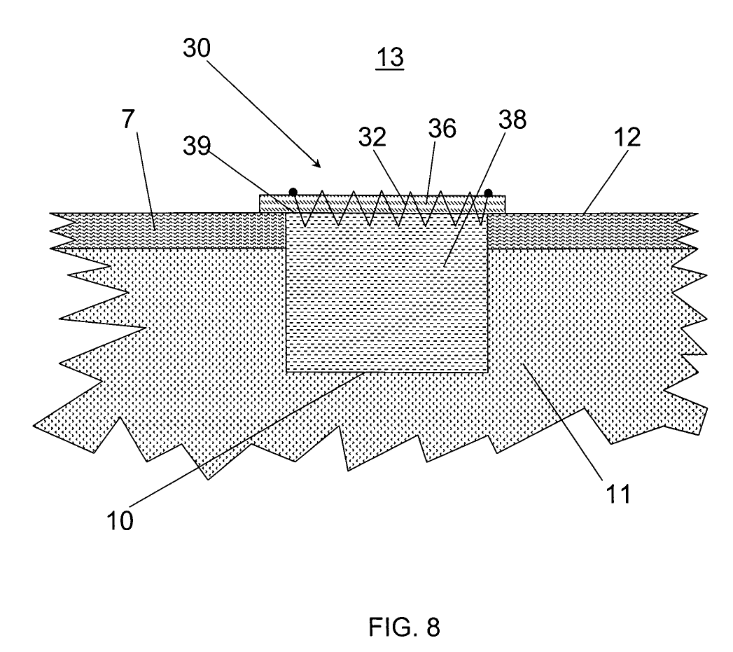

FIG. 8 shows an embodiment implant 30 wherein the cover 36 is sutured onto the scaffold 38 end surface 39 that would otherwise be exposed to the synovial cavity 13. Enough tension is applied to the suture material 32 to ensure that a sealed barrier is formed between the biochemical environment of the synovial cavity 13 and the surgically prepared defect 10. Suturing the cover 36 should place the cover 36 in alignment with or slightly below the upper surface 12 of hyaline cartilage 7 associated with the synovial cavity 13. Alternatively, and as shown in FIG. 8, in certain preferred embodiments the cover 36 may overlap the cartilage 7 around the defect 10. As in the other embodiments, the defect 10 may have a diameter that is slightly larger than, or equal to, the outermost diameter of the cartilage repair scaffold 38, such that when the implant 30 is inserted completely into the defect 10, the scaffold 38 is flush against with sub-chondral bone tissue 11. Suturing is performed in a manner that is known to one of ordinary skill in the art.

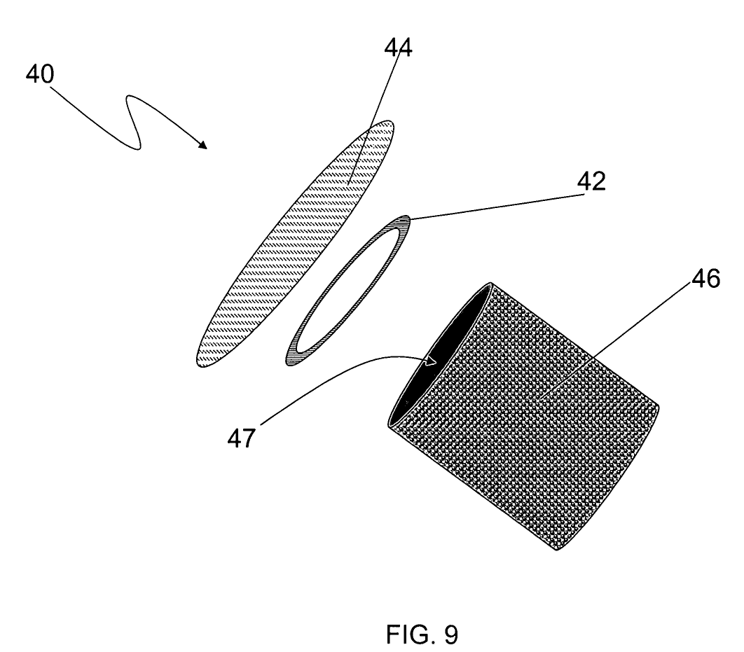

FIG. 9 shows an exploded view of an implant 40, where a more effective sealed barrier may be created by sandwiching a gasket 42 between irregular surfaces of the cover 44 and scaffold 46. If a cover 44 surface and a scaffold 46 end surface 47 are not capable of mating sufficiently to form a seal, a gasket 42 may be sandwiched between the two 44, 47 to fill such irregularities. The gasket 42 may further prevent leakage of materials from the synovial cavity into the defect while under compression between the cover 44 and scaffold 46.

For the implant 40, the scaffold 46 may be inserted into the defect. Subsequently, the gasket 42 may be placed about the scaffold 46 end surface 47 exposed to the synovial cavity. Axially fixing the cover 44 to the scaffold 46 end surface 47 will compress the gasket 42 between the two 44, 47, forming a sealed barrier between the defect and the materials of synovial cavity. As in the prior embodiment, the cover 44 may have a surface area that is larger than that of the end surface 47, so that the cover 44 overlaps the hyaline cartilage surrounding the defect. Alternatively, the cover 44 may snugly fit into the defect, laying flush with, or just slightly below, the top surface of the surrounding hyaline cartilage, so as to form a seal.

Materials for the Scaffold, Cover, Anchor, and Gasket

The implant, which includes the scaffold, cover, the various embodiments of the anchor, and optionally, the gasket, can be made from various materials which may include but are not limited to, ceramics, synthetic degradable polymers, synthetic non-degradable polymers, natural polymers, solid polymers and any combinations thereof.

Suitable non-limiting examples of ceramics include porous calcium phosphate such as, for example, hydroxyapatite (HA), tri-calcium phosphate (TCP) or any combination thereof, including, without limitations, approximately 30% HA and approximately 70% TCP. Calcium phosphate inherently binds certain growth factors to facilitate bone formation that synthetic polymers may not. It also has sufficient residence time in the patient to allow new bone or cartilage to form before it is degraded by the body.

Suitable non-limiting examples of synthetic biodegradable polymers include a-hydroxy acids, such as poly-lactic acid, polyglycolic acid, enantioners thereof, co-polymers thereof, polyorthoesters, and combinations thereof. Suitable non-limiting examples of synthetic non-biodegradable polymers include hydrogels such as PVA, delrin, polyurethane, polyethylene, co-polymers thereof and any combinations thereof.

Suitable non-limiting examples of natural polymers include, without limitations, collagen, elastin, silk, hyaluronic acid, chytosan, and any combinations thereof.

Since at least some of these polymers are generally hydrophobic, it may be advantageous to add compounds which increase the hydrophilic properties of these polymers and thus increase interactions between intercellular fluids of the sub-chondral bone tissue and hyaline cartilage and the implant. Suitable compounds include, without limitation, surfactants. Preferably, the surfactants are physiological surfactants, including, without limitation, non-toxic anionic, cationic, amphoteric or nonionic surfactants compatible with a bioactive agent and the materials forming the implant. Specific examples of such surfactants include, without limitation, metal soaps of fatty acids, alkyl aryl sulfonic acids, linear aklylbenzene sulfonates, alky sulfates, alcohol ethoxylates, alcohol ethoxy sulfates, alkylphenol ethoxylates, alpha olefin sulfonates, secondary alkane sulfonates, and alpha olefin sulfonates, as disclosed in U.S. Pat. No. 5,935,594 (Ringeisen), incorporated herein by reference in its entirety.

Methods for producing solid polymers are described, for example, in U.S. Pat. No. 5,290,494 (Coombes) incorporated herein by reference in its entirety. Generally, these methods involve the steps of: (1) polymer dissolution in a solvent; (2) casting the solution in a mold; (3) gel formation in situ; (4) removal of the shaped gel from the mold; and (5) drying to obtain a solid material in relatively thick sections.

Porous Cartilage Repair Scaffold

The cartilage repair scaffold can be porous, i.e. the scaffold's chemical structure may have a porous uniform, or possibly a glass-like, arrangement about its surfaces such that bone or cartilage tissue can easily penetrate beyond the outer surfaces of the scaffold and into the scaffold itself. However, it will be appreciated that the scaffold may be either porous or non-porous.

Having a porous cartilage repair scaffold may promote the ingrowth of bone and cartilage tissue into the implant, which may help to transfer load from the implant to newly formed sub-chondral bone and cartilage tissue that is in contact with the implant. Exterior pores of the implant may enhance the ability of cell attachment and thus allow for cellular migration and overgrowth of bone and cartilage tissue layers. The pores may be sized to maintain the mechanical strength of the scaffold. Although porosity of the scaffold may vary, the pores typically range from 10 μm to 500 μm.

Forming an implant with pores can be achieved by many methods. Crystals or powders, including but not limited to, sucrose, salt, calcium carbonate or sodium bicarbonate may be added during the molding process of an implant made of a synthetic polymer. The crystal or powder additive will embed into chemically bonded structure of the implant and, upon drying or dissolution of the implant, leave the implant in a porous state. A porous scaffold can also be created via solvent sublimation methods known in the art. An implant with a porous exterior may be accomplished by surface treatment of the implant with a plasma, including but not limited to a hydrogen peroxide plasma, or by milling. It is also within the scope of the invention to have the cover, scaffold, anchor, or the gasket made from polymeric fibers that are welded together by crossing, solvents, or heat.

Additives Associated with the Cover, Scaffold, or the Gasket

The exterior pores allow for optimal loading with bioactive agents, such as, for example, growth factors or cells, antibiotics, analgesics, radiocontrast agents, porogens, anti-inflammatory agents and the like. Preferably, the cover or the scaffold are associated with bone or cartilage inducing compounds at a concentration that is effective to induce the formation of cells that promote new bone or new cartilage tissue. The concentration of these compounds is such that new tissue is introduced at the site of the defect.

Suitable bioactive agents include, without limitation, growth factors (including osteogenic and chondrogenic agents), anti-inflammatory agents, pain-reducing agents, antibiotics, cells, and any combinations thereof.

Other suitable bioactive agents include, without limitation, BMP-1, BMP-2, rhBMP-2, BMP-3, BMP-4, rhBMP-4, BMP-5, BMP-6, rhBMP-6, BMP-7[OP-1], rhBMP-7, BMP-8, BMP-9, BMP-10, BMP-11, BMP-12, BMP-13, BMP-14, BMP-15, BMP-16, BMP-17, BMP-18, Growth and Differentiation Factors, GDF-5, Cartilage Derived Morphogenic Proteins, LIM mineralization protein, platelet derived growth factor (PDGF), transforming growth factor α, (TGF-α), insulin-related growth factor-I (IGF-I), insulin-related growth factor-II (IGF-II), fibroblast growth factor (FGF), beta-2-microglobulin (BDGF II), and rhGDF-5.

Suitable antibiotics include, without limitation nitroimidazole antibiotics, tetracyclines, penicillins, cephalosporins, carbopenems, aminoglycosides, macrolide antibiotics, lincosamide antibiotics, 4-quinolones, rifamycins and nitrofurantoin. Suitable specific compounds include, without limitation, ampicillin, amoxicillin, benzylpenicillin, phenoxymethylpenicillin, bacampicillin, pivampicillin, carbenicillin, cloxacillin, cyclacillin, dicloxacillin, methicillin, oxacillin, piperacillin, ticarcillin, flucloxacillin, cefuroxime, cefetamet, cefetrame, cefixine, cefoxitin, ceftazidime, ceftizoxime, latamoxef, cefoperazone, ceftriaxone, cefsulodin, cefotaxime, cephalexin, cefaclor, cefadroxil, cefalothin, cefazolin, cefpodoxime, ceftibuten, aztreonam, tigemonam, erythromycin, dirithromycin, roxithromycin, azithromycin, clarithromycin, clindamycin, paldimycin, lincomycirl, vancomycin, spectinomycin, tobramycin, paromomycin, metronidazole, tinidazole, ornidazole, amifloxacin, cinoxacin, ciprofloxacin, difloxacin, enoxacin, fleroxacin, norfloxacin, ofloxacin, temafloxacin, doxycycline, minocycline, tetracycline, chlortetracycline, oxytetracycline, methacycline, rolitetracyclin, nitrofurantoin, nalidixic acid, gentamicin, rifampicin, amikacin, netilmicin, imipenem, cilastatin, chloramphenicol, furazolidone, nifuroxazide, sulfadiazin, sulfametoxazol, bismuth subsalicylate, colloidal bismuth subcitrate, gramicidin, mecillinam, cloxiquine, chlorhexidine, dichlorobenzylalcohol, methyl-2-pentylphenol or any combination thereof.

Suitable anti-inflammatory compounds include the compounds of both steroidal and non-steroidal structures.

Suitable non-limiting examples of steroidal anti-inflammatory compounds are corticosteroids such as hydrocortisone, cortisol, hydroxyltriamcinolone, alpha-methyl dexamethasone, dexamethasone-phosphate, beclomethasone dipropionates, clobetasol valerate, desonide, desoxymethasone, desoxycorticosterone acetate, dexamethasone, dichlorisone, diflorasone diacetate, diflucortolone valerate, fluadrenolone, fluclorolone acetonide, fludrocortisone, flumethasone pivalate, fluosinolone acetonide, fluocinonide, flucortine butylesters, fluocortolone, fluprednidene (fluprednylidene) acetate, flurandrenolone, halcinonide, hydrocortisone acetate, hydrocortisone butyrate, methylprednisolone, triamcinolone acetonide, cortisone, cortodoxone, flucetonide, fludrocortisone, difluorosone diacetate, fluradrenolone, fludrocortisone, diflurosone diacetate, fluocinolone, fluradrenolone acetonide, medrysone, amcinafel, amcinafide, betamethasone and the balance of its esters, chloroprednisone, chlorprednisone acetate, clocortelone, clescinolone, dichlorisone, diflurprednate, flucloronide, flunisolide, fluoromethalone, fluperolone, fluprednisolone, hydrocortisone valerate, hydrocortisone cyclopentylpropionate, hydrocortamate, meprednisone, paramethasone, prednisolone, prednisone, beclomethasone dipropionate, triamcinolone. Mixtures of the above steroidal anti-inflammatory compounds can also be used.

Non-limiting example of non-steroidal anti-inflammatory compounds include nabumetone, celecoxib, etodolac, nimesulide, apasone, gold, oxicams, such as piroxicam, isoxicam, meloxicam, tenoxicam, sudoxicam, and CP-14,304; the salicylates, such as aspirin, disalcid, benorylate, trilisate, safapryn, solprin, diflunisal, and fendosal; the acetic acid derivatives, such as diclofenac, fenclofenac, indomethacin, sulindac, tolmetin, isoxepac, furofenac, tiopinac, zidometacin, acematacin, fentiazac, zomepirac, clindanac, oxepinac, felbinac, and ketorolac; the fenamates, such as mefenamic, meclofenamic, flufenamic, niflumic, and tolfenamic acids; the propionic acid derivatives, such as ibuprofen, naproxen, benoxaprofen, flurbiprofen, ketoprofen, fenoprofen, fenbufen, indopropfen, pirprofen, carprofen, oxaprozin, pranoprofen, miroprofen, tioxaprofen, suprofen, alminoprofen, and tiaprofenic; and the pyrazoles, such as phenylbutazone, oxyphenbutazone, feprazone, azapropazone, and trimethazone.

The various compounds encompassed by this group are well-known to those skilled in the art. For detailed disclosure of the chemical structure, synthesis, side effects, etc. of non-steroidal anti-inflammatory compounds, reference may be had to standard texts, including Anti-inflammatory and Anti-Rheumatic Drugs, K. D. Rainsford, Vol. I-III, CRC Press, Boca Raton, (1985), and Anti-inflammatory Agents, Chemistry and Pharmacology 1, R. A. Scherrer, et al., Academic Press, New York (1974), each of which is incorporated herein by reference.

Mixtures of these non-steroidal anti-inflammatory compounds may also be employed, as well as the pharmologically acceptable salts and esters of these compounds.

In addition, so-called “natural” anti-inflammatory compounds may be useful in methods of the disclosed invention. Such compounds may be obtained as an extract by suitable physical or chemical isolation from natural sources (e.g., plants, fungi, and by-products of microorganisms).

Suitable non-limiting examples of such compounds include candelilla wax, alpha bisabolol, aloe vera, Manjistha (extracted from plants in the genus Rubia, particularly Rubia Cordifolia), and Guggal (extracted from plants in the genus Commiphora, particularly Commiphora Mukul), kola extract, chamomile, sea whip extract, compounds of the Licorice (the plant genus/species Glycyrrhiza glabra) family, including glycyrrhetic acid, glycyrrhizic acid, and derivatives thereof (e.g., salts and esters).

Suitable salts of the foregoing compounds include metal and ammonium salts. Suitable esters may include C2-C24 saturated or unsaturated esters of the acids, preferably C10-C24, more preferably C16-C24. Specific examples of the foregoing may include oil soluble licorice extract, the glycyrrhizic and glycyrrhetic acids themselves, monoammonium glycyrrhizinate, monopotassium glycyrrhizinate, dipotassium glycyrrhizinate, 1-beta-glycyrrhetic acid, stearyl glycyrrhetinate, and 3-stearyloxy-glycyrrhetinic acid, and disodium 3-succinyloxy-beta-glycyrrhetinate.

Generally, anti-inflammatory non-steroidal drugs are included in the definition of pain-reducing agents because they provide pain relief. In addition, suitable pain-reducing agents may include other types of compounds, such as, for example, opioids (such as, for example, morphine and naloxone), local anaesthetics (such as, for example, lidocaine), glutamate receptor antagonists, α-adrenoreceptor agonists, adenosine, canabinoids, cholinergic and GABA receptors agonists, and different neuropeptides. A detailed discussion of different analgesics is provided in Sawynok et al., (2003) Pharmacological Reviews, 55:1-20, the contents of which are incorporated herein by reference.

All publications cited in the specification, both patent publications and non-patent publications, are indicative of the level of skill of those skilled in the art to which this invention pertains. All these publications are herein fully incorporated by reference to the same extent as if each individual publication were specifically and individually indicated as being incorporated by reference.

Dosage of Additives

Typically, the additives will total less than 1% to 10% by weight of the implant. The additives can be added to the implant during fabrication or after-coated about the surfaces of the implant. If the additives are added to the implant during fabrication, then they may be time released as the implant biodegrades.

Although 0.05 mg of a growth factor (BMP, for example) per gram of osteoconductive material, for example purified collagen and a biphasic calcium phosphate (BCP), is an amount sufficient to heal bone defects, the dose of growth factor required to effect osteo-induction is generally more. Accordingly about 0.1 mg to about 3 mg BMP, for example/g of osteo-conductive carrier is a preferred range. One example embodiment of the present invention comprises between about 2 mg and about 3 mg per gram (/g), e.g., about 2.5 mg protein /g of a osteo-conductive material.

### EXAMPLE 1

### Cartilage Repair Implant

Having generally described the implant, the following specific example is offered for purposes of illustration and only for illustration. No intention to limit the invention should be inferred. An implant for cartilage repair in keeping with the present disclosure may be prepared as follows:

The implant is manufactured by dissolving PLGA polymer in a solvent and adding 50% by wt. biphasic calcium phosphate particles (100-250 microns in diameter). This mixture is poured into large flat trays 20 mm in depth. These trays are placed into ovens to drive off the solvent creating a highly porous structure.

From these large porous PLGA/BCP sheets, 4-15 mm diameter plugs are cored and then cut to a desired 10-15 mm lengths. Similarly, porous collagen sheets 2-3 mm thick are made by pouring collagen slurry into trays and freeze drying under vacuum conditions. 4-15 mm diameter plugs are cut from the large sheet. Separately, 100-500 micron thick impermeable sheets of collagen membrane are made by pouring a collagen slurry into flat trays and thermal cross-linking in an oven at low temperature. Circular pieces of the collagen sheets 2-5 mm larger than the PLGA/BCP plugs are cut from the large collagen sheets.

A collagen slurry is then applied to the top surface of the PLGA/BCP plugs and one side of the circular collagen sheets to glue the porous collagen plugs to the porous PLGA/BCP plugs and the impermeable collagen membrane to the porous collagen layer. The resulting three layer structure is finally thermally cross-linked in an oven at low temperature. At the time of implantation, 1 mg of 1.5 mg/ml rhBMP-2 solution, the anabolic agent that promotes bone ingrowth into the lower subchondral bone area and cartilage into the upper cartilage layer, is added to the porous PLGA and collagen layers. The plug is then press fit into a prepared hole within the surface of the damaged cartilage.

The detailed description and example are not intended to limit the scope of the invention. One of ordinary skill in the art will appreciate that descriptions of the present invention are merely illustrations of preferred embodiments and that other arrangements may be devised without departing from the spirit and scope of the present invention as defined by the following claims.

What is claimed is:

1. An implant comprising:
(a) a cover; and
(b) a scaffold attached to the cover.

2. The implant of claim 1 wherein the cover is adapted to prevent influx of lubricin into the scaffold.

3. The implant of claim 2 wherein the cover is further adapted to prevent the influx of synovial fluid and inflammatory cytokines into the scaffold.

4. The implant of claim 2 wherein the cover has a larger surface area than an end surface of the scaffold to which the cover is attached.

5. The implant of claim 1 further comprising a means for axially fixing the cover to an end surface of the scaffold.

6. The implant of claim 5 wherein the means for axially fixing the cover to the end surface of the scaffold is selected from the set consisting of an anchor, a pin, an adhesive, a suture or combinations thereof.

7. The implant of claim 6 wherein the anchor axially extends from at least one surface of the cover.

8. The implant of claim 7 wherein an exterior surface of the anchor comprises a barb.

9. The implant of claim 7 wherein the anchor is centrally attached to at least one surface of the cover by a glue, a staple, a pin, or a combination thereof.

10. The implant of claim 7 wherein the anchor is centrally attached to the cover and axially penetrates through the end surface of the scaffold, and internally engages with the scaffold.

11. The implant of claim 1 wherein the cover and the scaffold are made from materials selected from the group consisting of collagen, hyaluronic acid, chitosan, natural polymers, aliphatic polyesters, polyorthoesters, polyanhydrides, polycarbonates, polyurethanes, polyamides, polyalkylene oxides, absorbable polymers, glasses or ceramics, autograft or allograft cartilage tissue, and any combinations thereof.

12. The implant of claim 1 wherein the implant is adapted to form a sealed barrier between an outer biochemical environment of a synovial cavity and an inner biochemical environment of a surgically prepared defect that extends from surfaces of hyaline cartilage into sub-chondral bone.

13. The implant of claim 12 wherein the cover is adapted to overlap the hyaline cartilage around the surgically prepared defect.

14. The implant of claim 1 wherein the cover is a sheet.

15. The implant of claim 1 wherein the scaffold is porous.

16. The implant of claim 1 wherein the scaffold is non-porous.

17. The implant of claim 1 wherein the scaffold is bio-resorbable.

18. The implant of claim 1 further comprising an effective amount of a biologically active additive.

19. The implant of claim 18 wherein the additive is selected from the group consisting of growth factors, antibiotics, analgesics, radiocontrast agents, porogens, anti-inflammatory agents and combinations thereof.

20. The implant of claim 19 wherein the growth factors are selected from the group consisting of BMP-1, BMP-2, rhBMP-2, BMP-3, BMP-4, rhBMP-4, BMP-5, BMP-6, rhBMP-6, BMP-7[OP-1], rhBMP-7, BMP-8, BMP-9, BMP-10, BMP-11, BMP-12, BMP-13, BMP-14, BMP-15, BMP-16, BMP-17, BMP-18, Growth and Differentiation Factors, GDF-5, Cartilage Derived Morphogenic Proteins, LIM mineralization protein, Nell-1 protein or peptide, platelet derived growth factor (PDGF), transforming growth factor α, (TGF-α), insulin-related growth factor-I (IGF-I), insulin-related growth factor-II (IGF-II), fibroblast growth factor (FGF), beta-2-microglobulin (BDGF II), rhGDF-5, and Nell-1.

21. The implant of claim 1 further comprising a gasket disposed between the scaffold and the cover.

22. The implant of claim 1 wherein the cover comprises at least a retaining ridge adapted to mechanically interact with the scaffold.

23. The implant of claim 1 wherein the cover and at least one scaffold end surface are fixed together with an adherent sealant.

24. The implant of claim 1 wherein the cover is an adherent sealant.

25. The implant of claim 24 wherein the adherent sealant is selected from the group consisting of cyanoacrylates, methylacrylates, octylacrylates, PEG, glycosaminoglysan, chitosan, collagen, hyaluronic acid, polyurethane solvents, and visible and UV activated adhesives.

26. The implant of claim 25 wherein the adherent sealant is adapted to adhere to surrounding hyaline cartilage tissue long enough to allow proper healing to occur in the areas of the scaffold in contact with both bone and cartilage tissue.

27. A method for repairing damaged cartilage comprising:
surgically removing damaged cartilage;
forming a hole in the area of the removed cartilage into sub-chondral bone tissue;
inserting a cartilage repair scaffold into the hole; and
providing a cover onto an end surface of the cartilage repair scaffold to prevent the ingress of synovial fluid or inflammatory cytokines into the cartilage repair scaffold.

28. The method of claim 27 wherein the cover further prevents the ingress of lubricin into the cartilage repair scaffold.

29. The method of claim 27 wherein the cover is attached to the cartilage repair scaffold prior to insertion of the cartilage repair scaffold into the hole.

30. The method of claim 27 wherein the cover is attached to the cartilage repair scaffold after insertion of the cartilage repair scaffold into the hole.

31. The method of claim 30 wherein the cover is an adhesive compound.

32. The method of claim 27 wherein the cover overlaps cartilage surrounding the hole.
